(19) United States
(12) Patent Application Publication
(10) Pub. No.: US 2012/0016478 A1
(43) Pub. Date: Jan. 19, 2012

(54) INTERVERTEBRAL IMPLANT WITH MULTI-PIECE END CAP

(75) Inventors: Bret M. Wilfong, Hernando, MS (US); Michael J. Merves, Memphis, TN (US)

(73) Assignee: Warsaw Orthopedic, Inc., Warsaw, IN (US)

(21) Appl. No.: 12/837,003

(22) Filed: Jul. 15, 2010

(51) Int. Cl. A61F 2/44

(52) U.S. Cl. 623/17.16

(57) ABSTRACT

Implants sized to be inserted into an intervertebral space between first and second vertebral members, and related methods. The spinal implant may have a body extending along a longitudinal axis with opposing first and second longitudinal ends. An end cap is mounted to the body at one of the ends and disposed longitudinally outboard of the corresponding end. The end cap comprises a first portion, a second portion, and an annular elastic element, such as an O-ring. The first and second portions are disposed circumferentially around the longitudinal axis. The annular elastic member circumferentially surrounds the first portion, the second portion, and the longitudinal axis. The annular elastic member applies a compressive force that urges the first and second portions together in a direction transverse to the longitudinal axis. The end cap may be flat or angled. Other implants and related methods are disclosed.

### BACKGROUND

The present application is directed to devices and methods for stabilizing vertebral members, and more particularly, to intervertebral implants and methods of use for replacing an intervertebral disc, vertebral member, or combination of both to distract and/or stabilize the spine.

The spine is divided into four regions comprising the cervical, thoracic, lumbar, and sacrococcygeal regions. The cervical region includes the top seven vertebral members identified as C1-C7. The thoracic region includes the next twelve vertebral members identified as T1-T12. The lumbar region includes five vertebral members L1-L5. The sacrococcygeal region includes nine fused vertebral members that form the sacrum and the coccyx. The vertebral members of the spine are aligned in a curved configuration that includes a cervical curve, thoracic curve, and lumbosacral curve. Intervertebral discs are positioned between the vertebral members and permit flexion, extension, lateral bending, and rotation.

Various conditions may lead to damage of the intervertebral discs and/or the vertebral members. The damage may result from a variety of causes including but not limited to a specific event such as trauma, a degenerative condition, a tumor, or infection. Damage to the intervertebral discs and vertebral members can lead to pain, neurological deficit, and/or loss of motion.

Various procedures include replacing the entirety or a section of a vertebral member, the entirety or a section of an intervertebral disc, or both. One or more replacement implants may be inserted to replace the damaged vertebral members and/or discs. The implants are configured to be inserted into the intervertebral space and contact against the remaining adjacent vertebral members. The implants reduce or eliminate the pain and neurological deficit, and increase the range of motion.

While a number of implant designs have been proposed, they have not proven ideal for all situations. As such, there remains a need for alternative intervertebral implant designs, particularly designs that allow for easy adaptation to spinal morphology.

### SUMMARY

The present application describes a spinal implant and associated method. In one embodiment, a spinal implant for insertion into an intervertebral space between first and second vertebral members comprises a body extending along a longitudinal axis with opposing first and second longitudinal ends. An end cap is mounted to the body at one of the ends and disposed longitudinally outboard of the corresponding end. The end cap comprises a first portion, a second portion, and an annular elastic element. The first and second portions are disposed circumferentially around the longitudinal axis. The annular elastic member, such as an O-ring, circumferentially surrounds the first portion, the second portion, and the longitudinal axis. The annular elastic member applies a compressive force that urges the first and second portions together in a direction transverse to the longitudinal axis. The end of the body having the end cap mounted thereto may comprise a plurality of projections projecting away from a center of the body, with at least some of the projections inter-engaged with the end cap to mount the end cap to the body.

In another embodiment, an intervertebral implant comprises a body extending along a longitudinal axis with opposing first and second longitudinal ends; the body comprising a first section that includes the first end and a second section that includes the second end; wherein the second section is adjustably longitudinally positionable relative to the first section while coupled thereto. The first end has a plurality of protrusions extending longitudinally away from the second end; at least two of the protrusions having undercuts. An end cap is mounted to the body at the first end and disposed opposite to the second section relative to the first section. The end cap comprises a first circumferential segment having a first lip, and a second circumferential segment distinct from the first segment and having a second lip. The first and second segments are coupled together and disposed circumferentially around the longitudinal axis, with the first lip engaging a first of the undercuts and the second lip engaging a second of the undercuts. The first and second segments define at least in part a longitudinally extending bore through the end cap. The end cap includes an annular elastic member that circumferentially surrounds the first segment, the second segment, and the longitudinal axis. The annular elastic member applies a compressive force that urges the first and second segments together in a direction transverse to the longitudinal axis so as to maintain the first and second lips in engagement with their respective undercuts. Sometimes, the first and second portions of the end cap are mirror images of each other, with each of the first and second portions extending for a circumferential arc of at least 90° relative to the longitudinal axis.

In another embodiment, a method of assembling a spinal implant comprises: providing a body extending along a longitudinal axis with opposing first and second longitudinal ends; the body comprising a first section that includes the first end and a second section that includes the second end; wherein the second section is adjustably longitudinally positionable relative to the first section while coupled thereto. An end cap having a first portion and a second portion distinct from the first portion is provided. The method includes mating the first portion of the end cap to the body at the first end of the body by moving the first portion transversely to the longitudinal axis. Thereafter, the second portion of the end cap is mated to the body at the first end of the body by moving the second portion transversely to the longitudinal axis. Thereafter, the first portion of the end cap is bound to the second portion of the end cap by circumferentially surrounding the first and second portions with an annular elastic member that urges the first and second portions together in a direction transverse to the longitudinal axis; wherein the annular elastic member circumferentially surrounding the first portion, the second portion, and the longitudinal axis.

The various aspects of the various embodiments may be used alone or in any combination, as is desired.

### BRIEF DESCRIPTION OF THE DRAWINGS

FIG. 1 is a perspective view of an implant according to one embodiment of the present invention.

FIG. 2 is a partially exploded view of the implant of FIG. 1.

FIG. 3 is a partially exploded view of a portion of the implant body and the corresponding end cap of FIG. 1.

FIG. 4 is a top view of FIG. 3 in the assembled condition.

FIG. 5 is a cross-section along line V-V of FIG. 4.

FIG. 6 shows an angled type end cap according to another embodiment of the present invention.

### DETAILED DESCRIPTION

The present application is directed to intervertebral implants for spacing apart vertebral members. The implant 20 is intended to be positioned within an intervertebral space formed between vertebral members. The implant 20 includes a body 30 and at least one end cap 50. The end cap 50 includes at least two distinct portions (or segments) 60,70 that are held together by an annular elastic element 80, such as an O-ring. The annular elastic element 80 urges the two portions 60,70 together, keeping the end cap 50 mounted to the body 30 as discussed further below. The implant 20 may also include a second end cap 51 positioned at the opposite end. The second end cap 51 may be the same or different than the first end cap 50.

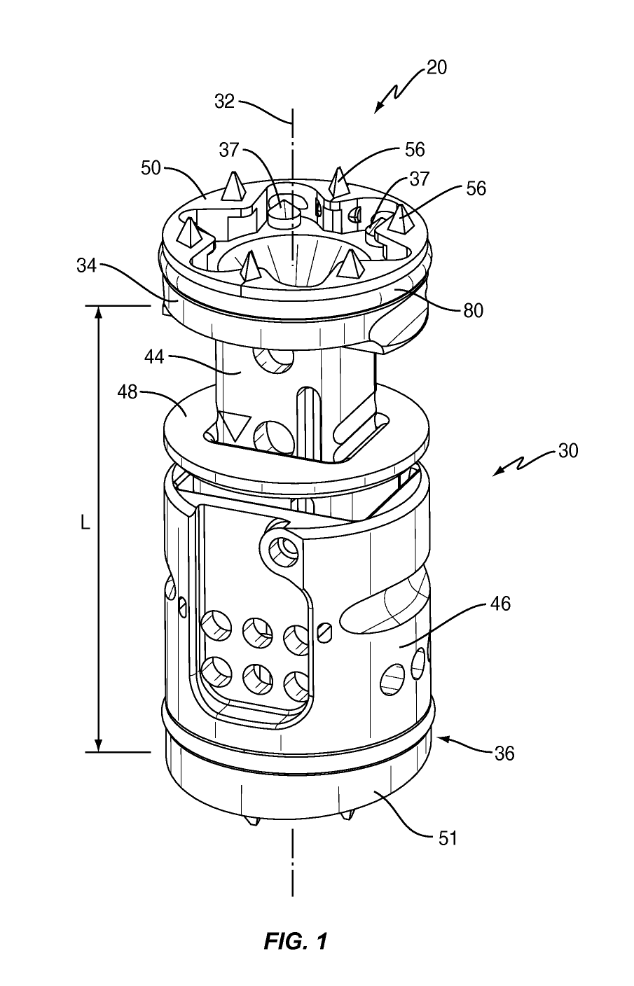

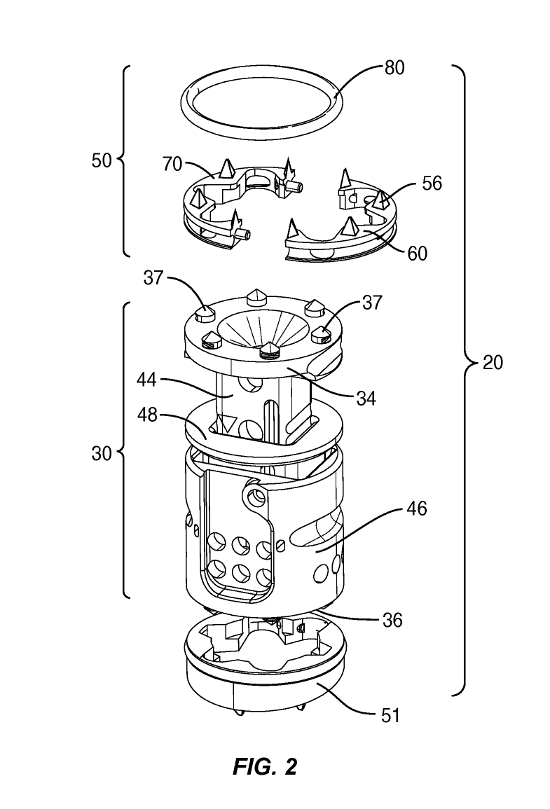

FIG. 1 shows one embodiment of the implant 20 in an assembled state. As more clearly shown in the partially exploded view of FIG. 2, the implant 20 includes a body 30 and at least one end cap 50. The body 30 in combination with the end cap 50 is sized to fit within the intervertebral space. The body 30 extends along a longitudinal axis 32 and includes a first end 34 and a second end 36. In some embodiments, the body 30 is of a fixed length along the longitudinal axis 32. In other embodiments, the length L of the body 30 along axis 32 is adjustable. For example, the body 30 may have two or more sections 44,46 that can be adjustably positioned relative to each other. The first section 44 may include the first end 34, and the second section 46 may include the second end 36, and the second section 46 may be telescopically received in the first section 44. A suitable locking mechanism 48, such as a pin or locking collar or the like, may be used to lock the relative positions of the first and second sections 44,46. For additional information about adjustable height implants, see, e.g., U.S. Patent Application Publications 2007/0255408; 2007/0255409; 2008/0114467; 2008/0177387; 2009/0164017, the disclosures of which are incorporated herein by reference in their entirety. In some embodiments, one or more apertures may extend through one or more sections of the body 30 to a hollow interior, such as for receipt of bone growth material. In some embodiments, the second end 36 may include a plurality of teeth designed to engage with the corresponding vertebral member.

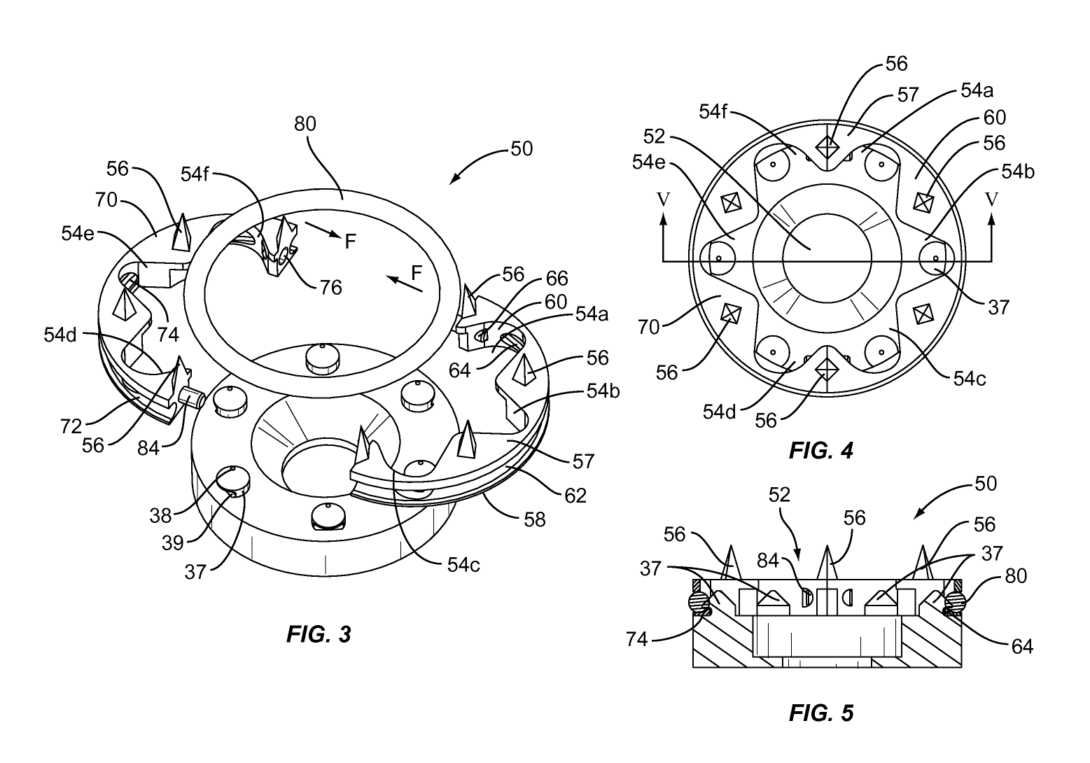

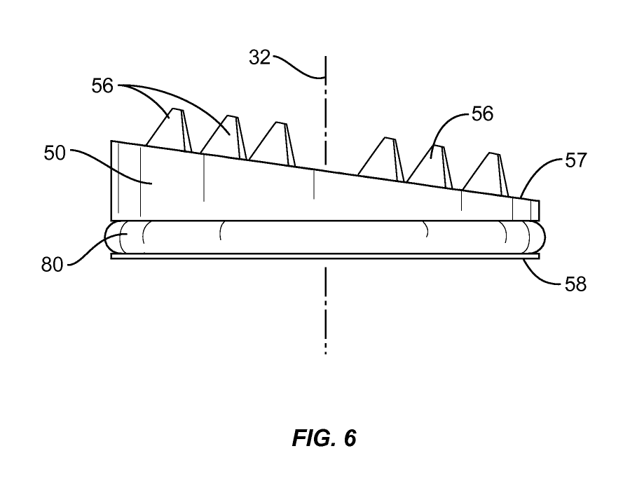

The first end 34 of the body 30 includes a plurality of protrusions 37 which extend outwardly away from the body 30, advantageously generally parallel to the longitudinal axis 32. The protrusions 37 are advantageously about six in number, but other numbers of protrusions 37 are within the scope of the invention. The protrusions 37 are advantageously arranged symmetrically about the longitudinal axis 32, at a common distance therefrom. The protrusions 37 may take the form of short posts, each with a pointed top 38. Advantageously, the protrusions 37 are all identical, but such is not required in all embodiments. Two or more of the protrusions 37 may include undercuts 39 that face radially outward, see FIG. 3 and FIG. 5.

End cap 50 is mounted to first end 34 of body 30. End cap 50 includes first portion 60, second portion 70, annular elastic member 80, and alignment pins 84. When assembled, end cap 50 forms an annular body of approximately the size of end 34 in a plane perpendicular to the longitudinal axis 32. The center of the annular body 30 includes a central bore 52 that is defined by the first and second portions 60,70. The central bore 52 advantageously includes a plurality of lobes 54, with the number of lobes 54 corresponding to the number of protrusions 37. The lobes 54 are advantageously symmetrically and evenly disposed about axis 32. The lobes 54 may all be identical, or the lobes 54 may have two or more configurations. For example, the middle lobe 54b of first portion 60 may be narrower than the outboard lobes 54a,54c, as shown in FIG. 4. Likewise, the middle lobe 54e of second portion 70 may be narrower than outboard lobes 54d,54f. The end cap 50 optionally includes teeth 56 projecting outward away from the body 30, advantageously generally parallel to the longitudinal axis 32. The teeth 56 are configured to bite into the corresponding vertebra, and may take any form known in the art.

First and second portions 60,70 are advantageously mirror images of each other; as such, the present discussion will focus on first portion 60, it being understood that the description may apply as well to second portion 70. First portion 60 forms a circumferential section of end cap 50. When there are two portions, the first portion 60 advantageously forms a 180° circumferential segment, but other amounts of circumferential sweep are within the scope of the present invention. In the illustrated embodiment, the first portion 60 has three recesses on its inner face which form three lobes 54 of the central bore 52. The center points of each of these three lobes 54 are advantageously located approximately 60° apart in circumferential sweep, with the middle lobe 54b disposed in the center of the first portion 60. A circumferential groove 62 extends around the exterior of first portion 60, between its upper surface 57 and its lower surface 58. The groove 62 is sized to receive the annular elastic member 80. Near the centers of the lobes 54, the groove 62 may open directly into the recesses forming the lobes 54. A lip 64 is formed in this location, which is sized to fit in the undercuts 39 of protrusions 37, see FIG. 5. The first portion 60 also includes two holes 66 that face the second portion 70. These holes 66 are sized to receive the alignment pins 84. The upper surface 57 of the first portion 60 may include one or more of the teeth 56.

Second portion 70 likewise includes a groove 72 and a retaining lip 74, and includes corresponding lobes 54d-f, if desired. In some embodiments, the second portion may likewise have holes 76 for the alignment pins 84. As mentioned above, the second portion 70 may be a substantial or complete mirror image of the first portion 60.

The annular elastic member 80 may take any suitable form. For example, the annular elastic member 80 may be an O-ring made from suitable biocompatible material(s). Alternatively, the annular elastic member 80 may be an elastic band of any suitable type, an end-to-end-joined coil spring, a cinched elastic cord, or other elastic member. The annular elastic member 80 should be sized to fit at least partially in grooves 62,72 and be sized to be under at least some tension when the end cap 50 is assembled.

The implant 20 may be assembled in a variety of ways. For example, alignment pins 84 may be placed in holes 66 of the first portion 60. The first portion 60 is then coupled to the body 30 by sliding the first portion 60 onto the first end 34 from a direction normal to the longitudinal axis 32. Such an approach allows the lips 64 of first portion 60 to slide under/into the corresponding undercuts 39 on the corresponding protrusions 37. It should be noted that in the embodiments where the protrusions 37 are evenly spaced about the first end 34, the first portion 60 of the end cap 50 need not be positioned in a single particular orientation relative to the body 30, but can be put at any one of a variety of functionally equivalent positions. For example, if there are six protrusions 37, then the first portion 60 of the end cap 50 can be joined to the body 30 in any one of six different relative positions. After the first portion 60 of the end cap 50 is joined to the body 30, the second portion 70 is then slid into position from the opposite direction, with the alignment pins 84 being received in the holes 76 of the second portion 70. As with the first portion 60, the lips 74 of the second portion 70 are captured in the corresponding undercuts 39. Joined in this manner, the first and second portions 60,70 are prevented from moving longitudinally away from the body 30 so as to become disconnected from the body 30. This type of engagement is maintained by urging the first portion 60 toward the second portion 70 (and vice-versa) so that the lips 64,74 remain captured by the undercuts 39. The force F to urge the first and second portions 60,70 toward each other is supplied by the tension in the annular elastic member 80.

In some embodiments, the second end 36 of body 30 may also have an end cap 51 mounted thereto. The second end cap 51 may be identical to the first end cap 50, or may be different, as is desired.

Once assembled, the implant 20 may be used in a conventional fashion. For example, the implant 20 may be placed into an intervertebral space that has been prepared in a conventional fashion, and then the length L or operational height of the implant 20 may be adjusted as desired for those embodiments having height adjustments. The teeth 56 on the end cap 50, if present, help secure the implant 20 in place by biting into the bone of the associated vertebra. Suitable bone growth material may then be added to the implant 20, and/or the bone growth material may be added to the implant 20 prior to implantation. The implantation is then terminated in a conventional fashion. It should be noted that the implants 20 described herein may be implanted within a living patient for the treatment of various spinal disorders. The implants 20 may also be implanted in a non-living situation, such as within a cadaver, model, and the like. The non-living situation may be for one or more of testing, training, and demonstration purposes.

In the embodiments discussed above, flat style end caps 50 have been used for illustrative purposes. When assembled to the body 30, the flat style end caps 50 have upper surfaces 57 and lower surfaces 58 that are generally parallel to each other, and disposed at a common angle to the longitudinal axis 32, such as perpendicular thereto. However, in some embodiments, one or both end caps 50 may be of an angular style, such that the upper surface 57 and lower surface 58 of the end cap 50 are not parallel, but are each disposed at respective different angles that are both transverse to the longitudinal axis 32. The respective angles may vary from each other by, for example, 4°-15° or other desired amounts.

The discussion above has assumed that the alignment pins 84 are distinct from the first and second portions 60,70. With such an arrangement, the first and second portions 60,70 can be, if desired, mirror images of each other. However, such is not required, and the alignment pins 84 can be made integral with one or both of the first and second portions 60,70. Further, while simple pins 84 have been used for illustrative simplicity, other alignment approaches, such as rails and grooves, tapered and interleaving projections/recesses, or the like could alternatively be used to help align the second portion 70 relative to the first portion 60.

The discussion above has also used a round shape for the end cap 50. However, the end cap 50 need not be round, and other regular shapes, such as octagonal, square, oval, football-like, or semi-symmetric or irregular shapes can alternatively be employed without departing from the present invention. Likewise, the discussion above has used an end cap 50 of two distinct peripheral portions 60,70 as an illustrative embodiment. However, the periphery of end cap 50 may be formed of more than two distinct peripheral portions, such as three, four, or more portions, without departing from the present invention. For example, the end cap 50 may be formed of four portions, each sweeping a peripheral arc of 90°. For such an arrangement, there should be at least four protrusions 37 with undercuts 39 disposed in the corresponding locations. The various peripheral portions help form the overall outer periphery of the end cap 50 and surround the central bore 52.

The discussion above has assumed that the end cap 50 will remain removable from the body 30, such as by removing the annular elastic member 80 and sliding the first and second portions 60,70 out of engagement with the body 30. However, in some embodiments, the end cap 50 may be permanently mounted to the body 30, such as through the addition of adhesives, welding, staking, locking detents, or other comparable means. If desired, the end cap 50 or body 30 may have roughed surfaces, which may facilitate locking together of the components.

The discussion above has sometimes used an O-ring as an example of the annular elastic member 80. While O-rings are typically circular in their undeformed state, both in top view and in cross-section, there is no requirement that the annular elastic member have such characteristics in all embodiments. Indeed, the annular elastic member 80 may have any suitable cross-sectional shape, such as trapezoidal, oval, octagonal, star, or any other desired shape including irregular shapes. Likewise, the annular elastic member 80, in its undeformed (e.g., unloaded) state, may have a shape in top view that is oval, octagonal, square, or any other desired shape that loops back on itself, including irregular shapes.

The components described above may be made from any suitable biocompatible material known in the art, such as titanium and its alloys, polymers such as PEEK or silicones, etc. By way of non-limiting example, the annular elastic element 80 may be made from silicone or stainless steel, and may advantageously be superelastic if desired.

Spatially relative terms such as “under”, “below”, “lower”, “over”, “upper”, and the like, are used for ease of description to explain the positioning of one element relative to a second element. These terms are intended to encompass different orientations of the device in addition to different orientations than those depicted in the figures. Further, terms such as “first”, “second”, and the like, are also used to describe various elements, regions, sections, etc and are also not intended to be limiting. Like terms refer to like elements throughout the description.

As used herein, the terms “having”, “containing”, “including”, “comprising” and the like are open ended terms that indicate the presence of stated elements or features, but do not preclude additional elements or features. The articles “a”, “an” and “the” are intended to include the plural as well as the singular, unless the context clearly indicates otherwise.

The present invention may be carried out in other specific ways than those herein set forth without departing from the scope and essential characteristics of the invention. The present embodiments are, therefore, to be considered in all respects as illustrative and not restrictive, and all changes coming within the meaning and equivalency range of the appended claims are intended to be embraced therein.

What is claimed is:

1. A spinal implant for insertion into an intervertebral space between first and second vertebral members, comprising:
a body extending along a longitudinal axis with opposing first and second longitudinal ends;
an end cap mounted to the body at one of the ends and disposed longitudinally outboard of the corresponding end;
the end cap comprising:
a first portion;
a second portion distinct from the first portion and coupled to the first portion;
wherein the first and second portions are disposed circumferentially around the longitudinal axis;
an annular elastic member circumferentially surrounding the first portion, the second portion, and the longitudinal axis;
the annular elastic member applying a compressive force that urges the first and second portions together in a direction transverse to the longitudinal axis.

2. The implant of claim 1 wherein the annular elastic member is an O-ring.

3. The implant of claim 1 wherein the end of the body having the end cap mounted thereto comprises a plurality of projections projecting away from a center of the body; wherein at least some of the projections inter-engage with the end cap to mount the end cap to the body.

4. The implant of claim 1 wherein the body comprises first and second sections, with the first end associated with the first section and the second end associated with the second section; wherein the first section is displaceable relative to the second section to adjust the longitudinal length of the implant.

5. The implant of claim 1 wherein the first and second portions of the end cap each include an exteriorly facing groove; wherein the annular elastic member is disposed in the grooves of the first and second portions.

6. The implant of claim 1 wherein the end cap comprises a central opening having a plurality of lobes.

7. The implant of claim 6 wherein the lobes are symmetrically disposed relative to the longitudinal axis.

8. The implant of claim 7 wherein the lobes are of at least two different configurations, with lobes disposed directly across from one another with respect to the longitudinal axis being of the same configuration.

9. The implant of claim 1 wherein the end cap further comprises a plurality of teeth extending away from the body.

10. The implant of claim 1 wherein the end cap further comprises an alignment post and the second portion includes a recess for receiving the post; wherein the post extends into the recess.

11. The implant of claim 1 wherein the end cap further comprises a third portion distinct from both the first and second portions; wherein the third portion is coupled to at least one of the first and second portions; wherein the annular elastic member circumferentially surrounds the first, second, and third portions.

12. The implant of claim 1 wherein the end cap is removably mounted to the corresponding end of the body.

13. The implant of claim 1 wherein the end cap comprises an exterior face opposite the body, wherein the exterior face is disposed at a non-perpendicular angle relative to the longitudinal axis.

14. The implant of claim 1 wherein the first and second portions of the end cap are mirror images of each other.

15. The implant of claim 1 wherein the end cap further comprises an inner surface facing toward the body and an outer surface facing away from the body; wherein the inner and outer surfaces are disposed at a non-parallel angle relative to each.

16. An intervertebral implant, comprising:
a body extending along a longitudinal axis with opposing first and second longitudinal ends; the body comprising a first section that includes the first end and a second section that includes the second end; wherein the second section is adjustably longitudinally positionable relative to the first section while coupled thereto;
the first end comprising a plurality of protrusions extending longitudinally away from the second end; at least two of the protrusions having undercuts;
an end cap mounted to the body at the first end and disposed opposite to the second section relative to the first section;
the end cap comprising:
a first circumferential segment having a first lip;
a second circumferential segment distinct from the first segment and having a second lip;
wherein the first and second segments are coupled together and disposed circumferentially around the longitudinal axis, with the first lip engaging a first of the undercuts and the second lip engaging a second of the undercuts;
the first and second segments defining at least in part a longitudinally extending bore through the end cap;
an annular elastic member circumferentially surrounding the first segment, the second segment, and the longitudinal axis;
the annular elastic member applying a compressive force that urges the first and second segments together in a direction transverse to the longitudinal axis so as to maintain the first and second lips in engagement with their respective undercuts.

17. The implant of claim 16 wherein the first and second portions of the end cap are mirror images of each other, and wherein each of the first and second portions extend for a circumferential arc of at least 90° relative to the longitudinal axis.

18. The implant of claim 16 wherein the bore of the end cap comprises a plurality of lobes symmetrically disposed relative to the longitudinal axis; wherein the lobes are of at least two different configurations, with lobes disposed directly across from one another with respect to the longitudinal axis being of the same configuration.

19. A method of assembling a spinal implant for insertion into an intervertebral space between first and second vertebral members, comprising:
providing a body extending along a longitudinal axis with opposing first and second longitudinal ends; the body comprising a first section that includes the first end and a second section that includes the second end; wherein the second section is adjustably longitudinally positionable relative to the first section while coupled thereto;
providing an end cap having a first portion and a second portion distinct from the first portion;
mating the first portion of the end cap to the body at the first end of the body by moving the first portion transversely to the longitudinal axis;
thereafter, mating the second portion of the end cap to the body at the first end of the body by moving the second portion transversely to the longitudinal axis;
thereafter, binding the first portion of the end cap to the second portion of the end cap by circumferentially surrounding the first and second portions with an annular elastic member that urges the first and second portions together in a direction transverse to the longitudinal axis; wherein the annular elastic member circumferentially surrounding the first portion, the second portion, and the longitudinal axis.

20. The method of claim 19:
wherein the first end comprises a plurality of protrusions extending longitudinally away from the second end; at least two of the protrusions having undercuts;
wherein each of the first and second portions comprise a respective lip;
wherein the mating the first portion of the end cap to the body comprises sliding the lip of the first portion into engagement with a first of the undercuts;
wherein the mating the second portion of the end cap to the body comprises sliding the lip of the second portion into engagement with a second of the undercuts.
